(19) United States
(12) Patent Application Publication
(10) Pub. No.: US 2022/0202312 A1
(43) Pub. Date: Jun. 30, 2022

(54) Respiratory Biofeedback-Based Content Selection and Playback for Guided Sessions and Device Adjustments

(71) Applicant: Auralab Technologies Incorporated, Ypsilanti, MI (US)

(72) Inventors: Robert Alexander, Detroit, MI (US); Benjamin Collins, Ypsilanti, MI (US)

(21) Appl. No.: 17/137,965

(22) Filed: Dec. 30, 2020

(51) Int. Cl. A61B 5/08; A61B 5/00; G16H 20/70

(52) U.S. Cl. CPC A61B 5/0816; G16H 20/70; A61B 5/7207; A61B 5/486

(57) ABSTRACT

Respiratory biofeedback-based content selection and playback includes using data obtained using one or more sensors of a mobile device running a software application to produce a respiratory data stream of a user of the software application and select content to output to the user. The respiratory data stream represents various respiratory information of the user and is processed to determine a user state of the user. Certain content is selected based on the user state and based on a defined respiratory objective of a guided session being run using the software application. Initial content previously output to the user during the guided session may then be adjusted by outputting the selected content to the user. A change in the user state resulting from the outputting of the selected content may then be used to determine a progress of the user toward achieving the defined respiratory objective of the guided session.

### TECHNICAL FIELD

This disclosure relates to respiratory biofeedback-based content selection and playback for guided sessions and device adjustments, namely, to a software application for monitoring user respiratory information and using same to guide activity and/or control connected devices.

### BACKGROUND

Biofeedback generally refers to the process of using monitored bodily functions and responses, so as to control those functions and responses. Conventional biofeedback systems measure or acquire physiological data, translate the physiological data into a digital or analog signal, and provide a processed version of this signal to the user using one or more sensory modalities (e.g., audio feedback, video feedback, haptic feedback, or the like).

### SUMMARY

Disclosed herein are, inter alia, systems and techniques for respiratory biofeedback-based content selection and playback for guided sessions and device adjustments.

In an implementation, a method for respiratory biofeedback-based content selection and playback is provided. The method includes initiating a guided session at a software application running on a mobile device. Using data obtained using one or more sensors of the mobile device, a respiratory data stream representing a stream of respiratory information of a user of the software application is produced while the mobile device rests on the user. The respiratory data stream is processed to determine a user state of the user. Content is selected for output to the user based on the user state and based on a defined respiratory objective of the guided session. Initial content previously output to the user during the guided session is adjusted by outputting the selected content to the user. A progress of the user toward achieving the defined respiratory objective of the guided session is then determined based on a change in the user state resulting from the outputting of the selected content.

In another implementation, a method for respiratory biofeedback-based content selection and playback is provided. The method includes processing, during a guided session at a software application running on a mobile device, a respiratory data stream of a user of the software application to determine one or more of a respiratory curve, a respiratory stability, or a respiratory rate of the user. Based on the one or more of the respiratory curve, the respiratory stability, or the respiratory rate of the user, content to use to adjust initial content previously output to the user during the guided session is selected. The initial content is then adjusted according to the selected content by outputting the selected content during the guided session.

In yet another implementation, a method respiratory biofeedback-based content selection and playback is provided. The method includes producing, at a first time and using first data obtained using one or more sensors of a mobile device, a first respiratory data stream of a user of a software application running on the mobile device while the mobile device rests on the user and while the user participates in a guided session of the software application. The first respiratory data stream is processed to select first content to output to the user based on the guided session. The first content is then output for presentation to the user, wherein the first content is configured to change a user state of the user to a first state. At a second time after the first time, and using second data obtained using the one or more sensors of the mobile device, a second respiratory data stream of the user is produced while the mobile device rests on the user and while the user participates in the guided session. The second respiratory data stream is processed to select second content to output to the user based on the guided session. The second content is then output for presentation to the user, wherein the second content is configured to change the user state of the user from the first state to a second state.

### BRIEF DESCRIPTION OF THE DRAWINGS

This disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 shows a block diagram of an example of a device used for respiratory biofeedback-based content selection and playback.

FIG. 2 shows a block diagram of example functionality of a software application for respiratory biofeedback-based content selection and playback.

FIG. 3 shows a block diagram of examples of software modules used for determining and processing respiratory biofeedback information for a user of a software application.

FIG. 4 shows a block diagram of an example workflow for respiratory biofeedback-based content selection and playback.

FIG. 5 shows a block diagram an example of device adjustment of a connected device using a software application for respiratory biofeedback-based content selection and playback.

FIG. 6 shows a block diagram of an example of a multi-user system for respiratory biofeedback-based content selection and playback.

FIG. 7 shows a flowchart showing an example of a technique for respiratory biofeedback-based content selection and playback for a guided session.

FIG. 8 shows a flowchart showing an example of a technique for respiratory biofeedback-based content selection and playback for a device adjustment.

FIG. 9 shows a block diagram of an example internal structure of a computing device which may be used for respiratory biofeedback-based content selection and playback.

FIGS. 10A-B show example illustrations of a wearable application running on a wearable device.

### DETAILED DESCRIPTION

Mindful breathing is challenging and often overlooked, particularly in western cultures. Many are unaware that the decrease in blood-oxygenation from improper breathing can cause fatigue, stress on the heart and elevated cortisol levels. This in turn may lead to weight gain, erratic moods, and difficulty sleeping among other complications. It is underappreciated that the region of the brain responsible for breathing has direct, dramatic influence on higher-order brain function, and shallow breathing triggers the “fight-or-flight” response in the body. The net result is stress.

Existing approaches to facilitating proper breathing and deep slow breathing specifically have come up short of an adequate solution. One approach uses an instructional video that demonstrates proper placement of a user device on an abdominal area of a participant, and the breath of the participant triggers a series of pre-recorded audio files including ocean-wave like sounds and musical chords; however, this approach is limited in that audio files are only triggered at specific times, for example, when the participant inhales and exhales. The mere playback of pre-recorded content at a time when someone inhales and exhales is insufficient to facilitate proper breathing, and, further, fails to adequately process and understand respiratory qualities specific to the participant to determine how and when to interact with the participant.

Implementations of this disclosure address problems such as these by using a software application for respiratory biofeedback-based content selection and playback for guided sessions and device adjustments, which, in particular, enables experiences to be generated in real-time in a way that is customized to the evolving physiological, mental and affective state of the individual user, as determined in a biofeedback loop based on respiratory information collected for the user. Thus, the respiratory biofeedback aspect of this disclosure refers to the collection and use of respiratory information collected for a user of the software application to determine how to interact with the user, For example, the software application can determine to select or adjust content in the form of audio and/or visual content based on that respiratory information and then playback that selected or adjusted content to the user presented at the device running the software application, trigger of functionality of a connected device based on that respiratory information, or both. The respiratory information used by the software application in either case corresponds to respiratory cycles (RCs) of a user of the software application, in which a RC begin at the onset of inhalation, and terminates after exhalation has completed. The biofeedback loop evaluates RC data to implement the functionality of the software application, as disclosed herein.

A software application as disclosed herein may be used for guided sessions of one or more activity types, which use respiratory biofeedback for a user of the software application to configure the guided sessions in real-time. As used herein, a guided session refers to an activity in which a participant, that is, the user of the software application disclosed herein, is lead using cues intended to guide the actions actively or passively performed by the participant. For example, the cues may be or include spoken-guidance from a leader of the guided session, such as via a live stream or via a pre-recorded source. In another example, the cues may be or include audio guidance other than speech, such as music, tones, or other sounds. In yet example, the cues may be or include visual cues, such as images, animations, or videos. The cues, regardless of their form, are intended to guide user focus throughout a guided session, such as toward a goal of the guided session. Several types of guided session may be available, in which each type may have a different goal for user breath activity. A guided session may, for example, be a sleep induction session, a meditation session, a stress management session, a relaxation management session, a pain management session, a health and fitness session, or another session. Although certain types of guided sessions are specifically described in this disclosure, the implementations of this disclosure may also be performed with respect to other types of guided sessions.

For example, in a sleep induction session, audio and/or visual output may be presented to the user of the software application to assist the user in achieving deep relaxation, in which that output presented to the user is determined by and adjusted according to real-time respiratory biofeedback data collected from the user. In another example, in a pain management session, audio and/or visual output may be presented to the user of the software application to direct the focus of the user toward an area of his or her body which is in pain, such as by directing the user to “breathe into” this area with focused attention. In yet another example, in a stress or relaxation management session, audio and/or visual output may be presented to the user of the software application to direct the user to achieve and sustain for some amount of time a breathing pattern for reducing stress.

A guided session has a desired objective for the participant, which objective may generally be based on the type of the guided session. For example, the objective of a sleep induction session may be to cause the user to fall asleep, whereas the objective of a relaxation management session may be to cause the user to achieve a relaxed physical and/or emotional state. A guided session may be considered to have one or more phases, delineated by transition points, which transition the participant from an initial state at the beginning of the guided session to zero or more intermediate states and then to a final state at the end of the guided session, which end may either occur automatically at a certain time determined by the software application (e.g., based on the participant meeting the objective of the guided session) or manually by the participant's termination of the guided session.

The software application as disclosed herein may further, such as in addition to or instead of being used for a guided session, be used to trigger functionality of one or more connected devices based on respiratory biofeedback for the user of the software application. As used herein, a connected device refers to a device which is connected to a network and which has some kind of functionality which can be automatically and/or manually triggered over that network. For example, a connected device may be or refer to a smart light bulb which can be selectively turned on and off, and in some cases dimmed or flashed, based on commands transmitted to it or to a hub with which the smart light bulb is associated. In another example, a connected device may be or refer to a smart thermostat via which the temperature setting for some indoor environment may be adjusted based on commands transmitted to it or to a hub with which the smart thermostat is associated. Although certain types of connected devices are specifically described in this disclosure, the implementations of this disclosure may also be performed with respect to other types of connected devices.

The functionality of a connected device as is disclosed herein may be triggered based on respiratory biofeedback for a user of a software application. For example, certain events as may be derived from the respiratory biofeedback of the user of the software application may be defined to cause certain functionality of one or more connected devices to be performed. For example, respiratory biofeedback indicating that the user of the software application has entered a meditative state may be used by the software application to cause one or more smart light bulbs to either turn off or dim in brightness. In another example, respiratory biofeedback indicating that the user of the software application is close to entering a sleep state may be used by the software application to cause a smart thermostat to be adjusted to a slightly decreased temperature setting to better accommodate the user entering and remaining in the sleep state.

In some cases, connected device aspects of this disclosure may be combined with guided session aspects of this disclosure. For example, a software application as is disclosed herein may be used to trigger functionality of one or more connected devices as part a guided session, such as based on the type of guided session, an event occurring during a guided session, a transition point of a guided session, or a combination thereof. For example, in a guided session for sleep induction, the beginning of the guided session may trigger certain smart light bulbs to dim to a first brightness level and trigger a smart thermostat to adjust a temperature setting. After respiratory biofeedback of the user of the software application indicates that the user has entered a relaxed state, the software application may trigger those smart light bulbs to dim to a second brightness level lower than the first brightness level. Once the respiratory biofeedback of the user of the software indicates that the user has entered a sleep state, the software application may trigger those smart light bulbs to turn off.

Example use cases for implementations of respiratory biofeedback-based content selection and playback as disclosed herein include, but are not limited to, tucking the device into a waist band while riding an airplane, sitting in a passenger seat of a car, sitting on a recliner, sitting on an office chair, lying on a soft surface (e.g., a bed or hammock), and lying on a floor or ground. Different use cases for the device may use or otherwise be optimal with particular positions of the user. In some implementations, the operations of the device for respiratory biofeedback using audio parameter mapping may support changing between multiple different positions (e.g., if the user begins a session reclining and decides to shift to a fully supine position).

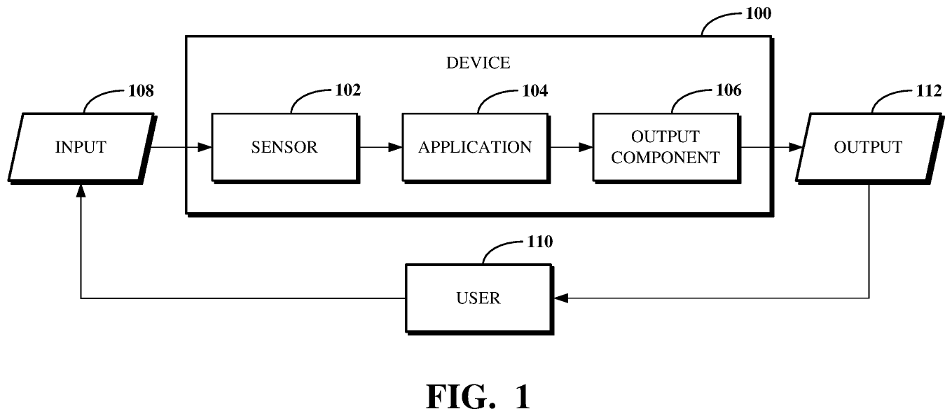

To further describe the implementations of the disclosure, reference is first made to a device which may be used therewith. FIG. 1 shows a block diagram of an example of a device 100 used for respiratory biofeedback-based content selection and playback. The device 100 includes a sensor 102, an application 104 (e.g., comprising instructions stored in a memory of the device 100 and executed, interpreted, or otherwise run using a processor of the device 100), and an output component 106. The sensor 102 receives input 108 from a user 110. The output component 106 is used to produce output 112 perceptible to the user 110.

The device 100 is a physical device which is capable of processing the input 108, which drives the output 112 to the user 110, thereby producing a respiratory biofeedback loop. In particular, where the input 108 relates to respiratory information of the user 110, the sensor 102 is used to produce a respiratory data stream (RDS) representing a stream of respiratory information of the user 110. For example, the respiratory information can indicate when the user inhales and/or exhales, how long the inhales and/or exhales are, how far apart in time the inhales and exhales are, and/or other information related to the respiration of the user or otherwise to the physiology of the user. The device 100 is ideally placed on the torso of the user 110, who may be sitting, reclined, or in a fully supine position. The device 100 may, for example, be a smartphone or other mobile device, which provides a convenient form-factor. Using a single device with these features may have advantages over other types of biofeedback that require separate sensors and processors.

The sensor 102 uses the input 108 to generate or otherwise derive the RDS. In particular, the sensor 102 acquires, derives, or otherwise determines data related to a movement and/or a position of the user 110, such as which may be used to derive a RC of the user 110. The sensor 102 may be an accelerometer, gyroscope, or other sensor capable of acquiring information related to position, movement, orientation, rotation, and/or acceleration of all or a portion of the body of the user 110. Alternatively, the sensor 102 may instead be another type of sensor, whether internal or external to the device 100, which gathers data related to subtle or gross positional and/or physiological changes associated with the respiratory of the user 110.

The application 104 is a software application which receives the output of the sensor 102 as input and performs real-time calculations using one or more modules, as disclosed herein, to determine respiratory biofeedback-based information for the user 110. The application 104 uses a set of values (e.g., the RDS and/or some or all downstream permutations of the RDS) stored in a memory of the device 100, such as within a short-term memory and/or a long-term memory, to determine an output.

The modules of the application 104 are software modules having functionality that includes but is not limited to the processing that takes place on the RDS. The software modules can include modules for preparing the output of the sensor 102 for use in determining respiratory biofeedback information of the user 110, for example, by one or more of data cleaning, data processing, parameter extraction, and/or parameter mapping. The software modules may additionally or instead include modules for using the determined respiratory biofeedback information of the user 110, for example, by one or more of user state detection, algorithmic guidance, parameter mapping, and/or state shifting, such as may be part of a technique associated with a guided session and/or triggering functionality of a connected device.

In some implementations, the application 104 may include one or more GUIs, such as by data and rendering instructions for presenting the user 110 with information used to interact with the application 104. For example, such a GUI may be used to start a new session or to make modifications to a current session (e.g., setting of preferences, adjustment of parameters such as master volume levels, play/pause controls, and some level of control over the underlying algorithms and the parameters they generate).

The output 112 is largely derived from the processed RDS, extracted parameters, and selected parameter mappings (which may, for example, be customized based on definitions of audio parameters selectable within the application 104) processed using the application 104. The output 112 may include, but is not limited to, audio and/or visual modalities. The respiration of the user 110 drives the RDS produced using the sensor 102 based on the output 112 and based on subsequent input 108. In particular, the user 110 receives the output 112, which in turn modulates the respiration of the user 110. These changes in respiration are passed back through the sensor 102, creating a biofeedback loop. The output component 106 includes one or more components or devices of the device 100 which are used to deliver the output 112 to the user 110 in a form perceptible by the user 110. For example, the output component 106 may be or include a display (e.g., an LCD, LED, CRT, or other display), a speaker, or another component or device capable of outputting audio and/or visual output for the user 110.

In some implementations, a secondary device may be used by the application 104 to produce and transmit, to the device 100 for use by the application 104, biometric user data associated with the user 110. The biometric user data generally refers to biometric data which is not directly related to the breath of the user 110. For example, the biometric user data may be or refer to heart rate data, blood oxygen level data, blood pressure data, neural activity data, or other data indicative of a physiology of the user 110. In some such implementations, the application 104 may use the biometric user data along with data produced using the sensor 102 to determine respiratory biofeedback information, such as which may be processed for content selection and playback as disclosed herein.

Examples of a secondary device as used herein may include, but are not limited to, a wearable device (e.g., a smart watch, a smart ring, a smart wristband, a heart rate monitor, a heart rate variability monitor, a blood pressure monitor, a blood oxygen level monitor, a sleep tracker, etc.), a device peripheral or accessory which includes one or more biometric sensors (e.g., headphones, a strap, a microphone, etc.), a computing device which includes one or more biometric sensors (e.g., a tablet computer, a mobile device other than the device 100, a laptop computer, etc.), or a device (e.g., a wearable device or computing device) or device peripheral or accessory that has access to current and/or historical data collected or otherwise measured using such sensors (e.g., a storage device or server device accessed to store, view, or process data in connection with a health or related application). The secondary device may be in communication with the device 100 using a wired or wireless approach. In some such implementations, multiple secondary devices may be used to process and provide biometric user data for use with the application 104.

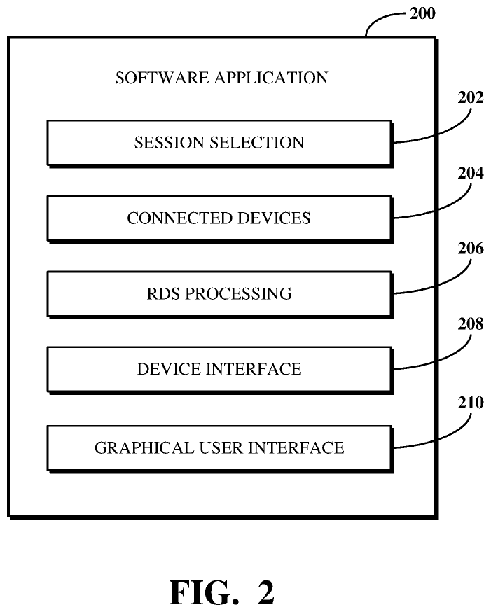

FIG. 2 shows a block diagram of example functionality of a software application 200 for respiratory biofeedback-based content selection and playback, which may, for example, be the application 104 shown in FIG. 1. As shown, the software application 200 includes session selection functionality 202, connected devices functionality 204, RDS processing functionality 206, device interface functionality 208, and graphical user interface (GUI) functionality 210.

The session selection functionality includes or otherwise refers to functionality for enabling a user of the software application 200 to select a session to participate in with the software application 200. The session may be a guided session, such as which may be lead live or by pre-recorded cues by one or more leaders, a freestyle session for which actions are left to the user of the software application 200 alone, or another type of session. The software application 200 may be preconfigured with a list of different session types, in which each or some of the sessions may be directed to different purposes for achieving different mindfulness, relaxation, health, or other objectives. The software application 200 may further be updated, so as to add, remove, or modify any pre-existing guided session information.

In some implementations, a user of the software application 200 may use the session selection functionality 202 to further configure a selected session with an additional layer of guidance. For example, the user may select a guided session for meditation and then further select additional guidance for deep relaxation, self-compassion, sleep induction, or another activity type. In some such implementations, objectives of both selected session types are considered, and user breath information is evaluated in view thereof, to determine content selection and parameter mapping options for the session as the session progresses.

The connected devices functionality 204 includes or otherwise refers to interactivity between the software application, such as during a session selected using the session selection functionality 202, and one or more connected devices. In particular, the software application 200 may maintain a list of connected devices which have been registered directly or indirectly with the software application 200. A connected device may be registered directly with the software application 200 by an application programming interface (API) of the software application 200 accessing and logging data associated with the connected device. A connected device may be registered indirectly with the software application 200 by linking a user account with a service with which that connected device is registered to the software application 200.

Functionality of a connected device registered with the software application 200 may be triggered as part of a session, such as a guided session or another session. In particular, a connected device may be selectively operated based on respiratory biofeedback information for the user of the software application 200 obtained during a session. For example, the respiratory biofeedback information may indicate that the user is not in a relaxed state. In such a case, it may be possible for the software application 200 to interface with a connected device located within the environment in which the user is present to cause some change in the operation of the connected device in order to cause the user to enter into a more relaxed state. For example, the software application 200 may directly or indirectly transmit a signal including a command for a smart lightbulb, as the connected device, to decrease in brightness.

The processing functionality 206 includes or otherwise refers to software modules used for respiratory biofeedback-based content selection and playback. In particular, the processing functionality 206 uses sensor data to determine respiratory biofeedback data of the user of the software application 200 and uses that respiratory biofeedback data for content selection and playback, such as part of a guided session and/or for device adjustment. The processing functionality 206 operates during a session, such as which may be selected using the session selection functionality 202, to evaluate user breath information and adjust the session according thereto, such as by the playback of content newly selected based on that breath information or the adjustment of already presented audio and/or visual content by parameter mapping. Because the respiratory biofeedback aspects of the software application 200 operate on a loop while a session remains active, the processing functionality 206 operates continuously while the session remains active.

The device interface functionality 208 includes or otherwise refers to backend software used to interface with components of the device which runs the software application 200. For example, the device interface functionality 208 may be software that uses various device drivers to obtain information from sensors of the device, such as either directly or via a memory buffer, to cause sensors to produce sensor data, to cause output components to present audio and/or visual output, to connect to a network, or perform other functionality associated with components of the device.

The GUI 210 functionality operates to render and output one or more GUIs to the display of a device which runs the software application 200. As used herein, a GUI can comprise part of a software GUI constituting data that reflect information ultimately destined for display. For example, the data can contain rendering instructions for bounded graphical display regions, such as windows, or pixel information representative of controls, such as buttons and drop-down menus. The rendering instructions can, for example, be in the form of HTML, SGML, JavaScript, Jelly, AngularJS, or other text or binary instructions for generating a GUI on a display that can be used to generate pixel information. A structured data output of one computing device can be provided to an input of the display so that the elements provided on the display screen represent the underlying structure of the output data.

In some implementations, the software application 200 may include further functionality beyond what is shown. For example, the software application 200 may include analytics functionality for analyzing respiratory biofeedback information collected from the user during a session performed using the software application 200. In some implementations, the software application 200 may use that analytics functionality to further track and analyze other biometric user data, such as which may be collected using a secondary device in communication with the device running the software application 200. For example, the software application 200 may in this way measure and monitor several aspects of the user's health beyond those associated with respiratory biofeedback, including, but not limited to, heart rate, heart rate variability, blood oxygen level, blood pressure, and sleep patterns. In some implementations, the biometric user data used by the software application 200 may be obtained from a local storage of the device running the software application 200 (e.g., the device 100), a secondary device, and/or a different device or sensor.

In some implementations, a wearable device, for example, a smart watch or another device capable of being worn by the user of the software application, may be used in connection with the software application 200 to implement additional functionality for a user of the software application 200 when that user wears the wearable device. For example, the wearable device may run a wearable device application which generates visual and/or haptic feedback, such as in the form of tactile pulses, and outputs same to the user. The feedback presented by the wearable device application may be used to facilitate breathing at a specific pace defined by the user, by the software application 200, or by another source.

The wearable device may be in communication with the device running the software application 200 so that the wearable device application can directly or indirectly adjust functionality of the software application 200. In this way, the wearable device application functionality enabling a specific breathing pace to be defined at wearable device may improve the breath performance of the user of the software application 200. In some such implementations, functionality of the wearable device application or portions thereof may be controlled using the software application 200.

For example, a user of the software application 200 may start a session (e.g., a guided session or another session) with the software application 200. Before the session begins, simultaneously with the start of the session, or shortly after the session begins, the wearable device may begin to output visual and/or haptic feedback intended to assist the user in breathing at a specific pace. The output may include a series of vibrations emanating from the wearable device at discrete time intervals so as to indicate times at which the user of the software application 200 should inhale and/or exhale during the session. Alternatively, or additionally, the output may include a visual display of current respiratory rate and/or respiratory stability for the user, for example, as a manner of real-time status of RDS processing for the user. Where respiratory information for the user is displayed, the respiratory information may be transmitted from the software application 200, collected at the wearable device itself, or otherwise obtained.

Breathing at a specific pace may enable a person to more consciously focus on a specific breath rhythm, and therefore to achieve a higher respiratory stability. Thus, the feedback generated by the wearable device application may better assist the user of the software application 200 in achieving a desired breath goal associated with the session.

In some such implementations, the wearable device application may include one or more customization options which may be configured to define the specific breathing pace. For example, the one or more customization options may correspond to one or more of a number of desired breaths per minute or other unit of time, a duration of a breath session, a specific inhale/exhale ratio based on a number of beats (e.g., represented as tactile vibrations) for each portion of the breath independently, the enabling of certain types of beats (e.g., transition beats for indicating the transition between inhalation and exhalation, primary beats which occur during each portion of the breath, etc.), or the like.

In some such implementations, the wearable device application may integrate with the software application 200 to enable the user of the software application 200 to control the software application 200 or portions thereof directly from the wearable device. For example, the wearable device application may include functionality to start a session with the software application 200, to configure customization options used to define the specific breathing pace for the session, and/or to otherwise interact with functionality of the software application 200. This may be particularly useful in cases where the mobile device of the user of the software application 200 is resting on or otherwise against the user, for example, by providing a second device usable to control inputs without disrupting the operation of the mobile device relative to the software application 200.

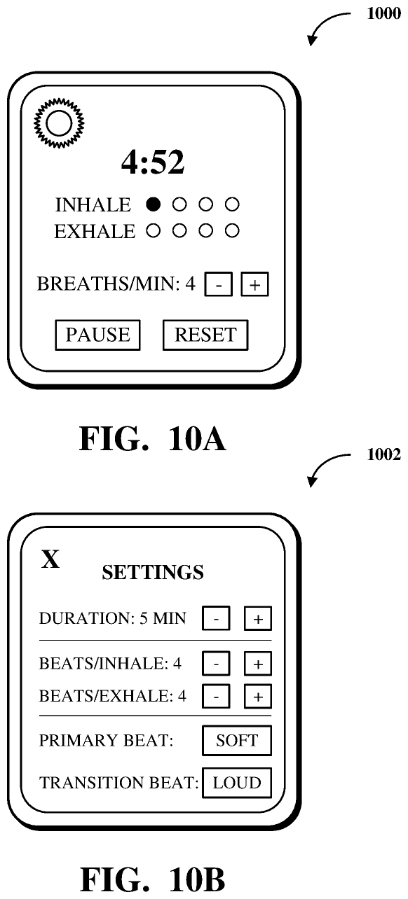

Example illustrations of a wearable application running on a wearable device are shown in FIGS. 10A-B. Referring first to FIG. 10A, a first GUI 1000 is shown. The first GUI 1000 includes elements indicating an amount of time which has elapsed in a current session, a number of inhales and exhales to be completed by the user within one minute, a number of breaths (e.g., in which each breath includes one inhale and one exhale) per minute for the user to complete which may be configurable using interactive elements to increment that number up or down, an interactive element to pause the current session, and an interactive element to reset the elapsed time for the current session. In the upper left corner of the first GUI 1000 is a gear element that, when interacted with, renders a second GUI 1002 shown in FIG. 10B. The second GUI 1002 includes interactive elements for configuring a duration of a current session, a number of beats per inhale, a number of beats per exhale, an intensity of a primary beat, and an intensity of a transition beat. Implementations of a wearable application as disclosed herein may include features instead of or in addition to what is shown in FIGS. 10A-B.

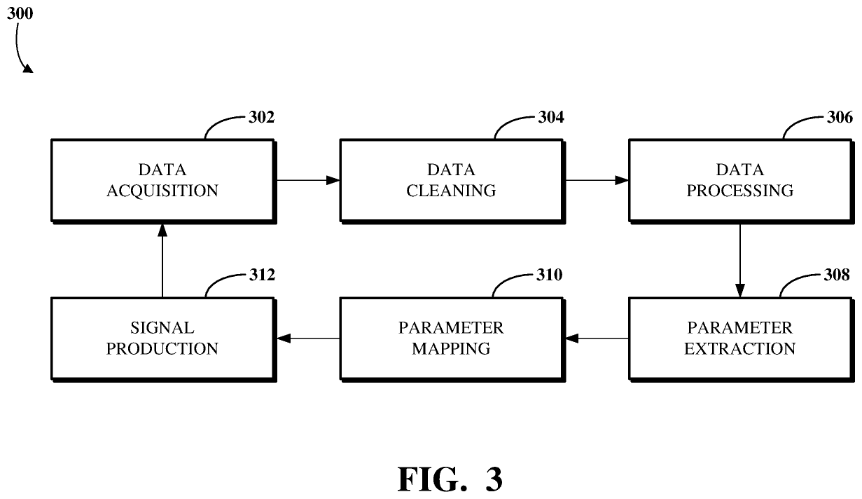

FIG. 3 shows a block diagram of examples of software modules 300 used for determining and processing respiratory biofeedback information for a user of a software application, for example, the software application 200 shown in FIG. 2. For example, the software modules 300 may represent some or all of the functionality of the RDS processing functionality 206 shown in FIG. 2. The software modules 300 present a high-level overview of the methodology of this disclosure and the data-flow employed within a device (e.g., the device 100 shown in FIG. 1) at which the software modules 300 are executed, interpreted, or otherwise run. The software modules 300 include a data acquisition module 302, a data cleaning module 304, a data processing module 306, a parameter extraction module 308, a parameter mapping module 310, and a signal production module 312.

The data acquisition module 302 receives or produces a raw RDS using data retrieved from or using a sensor (e.g., the sensor 102 shown in FIG. 1), as driven by the RC of a user (e.g., the user 110 shown in FIG. 1). This data may include a positional component, a rate of change component, and/or other components. For example, the data acquisition module 302 can be used to obtain or otherwise identify unfiltered accelerometer or gyroscope data. The unfiltered accelerometer or gyroscope data may, for example, be driven by a breath of the user of the device, such as based on movements of the user measured while the device rests on the user. The device resting on the user should be understood to include or otherwise refer to the device being set about a body of the user, for example, on top of or otherwise against a part of the body of the user (e.g., his or her chest or abdomen), in which case a sensor of the device should ideally be able to measure subtle changes in position with a high degree of accuracy. Additionally, a high sampling rate (e.g., 1-20 ms) may be more ideal for temporal synchronization between data input and the final output.

The data cleaning module 304 filters the raw RDS data received or produced using the data acquisition module 302, so as to remove noise and smooth subtle undesirable variances in the RDS. For example, the raw RDS data can be processed using a smoothing function such as a filter (e.g., a low-pass filter (LPF), a band-pass filter, or another filter) to remove high-frequency variance and/or noise. The smoothing filter is applied against the RDS to thus produce filtered (e.g., smoothed) data. The filtered data represents a denoised version of the data of the RDS as it was originally captured using the sensor of the mobile device.

In some implementations, the particular processing performed using the data cleaning module 304 may be based on an environment in which the user of the software application is located while running the software application. For example, additional denoising operations may be performed when the user is a passenger in a vehicle such as a car or airplane as compared to when the user is on a bed or other item of furniture. The denoising may be performed by using a function defined for the given environment in which the user is located, so as to account for unexpected movements of the user (e.g., arising from airplane turbulence or bumps in the road). For example, accelerometer data may be leveraged to determine a baseline signal to motion noise, and that baseline signal may be used to identify and thus remove noise introduced by the specific environment of the user. In some such implementations, the user may manually indicate the environment in which he or she is located within the software application. In other such implementations, the software application may derive a location of the user, and hence an environment in which the user is located, such as using positional and/or motion information obtained from a geolocation system, data captured within the environment (e.g., image and/or audio data captured using one or more sensors of the device running the software application), or other information. In some implementations, baseline data indicative of standard noise for a given environment may be stored at the application and used as a model for denoising, for example, in addition to or instead of a separate and new baseline being determined at the time a session is performed by the user. In some implementations, a filtering intensity of the data cleaning module 304 may be selectively configured by a user of the software application, such as via a GUI of the software application. For example, the software application may be enable the user thereof to configure the filtering intensity according to sensitivities present within the specific environment of the user.

The data processing module 306 processes the filtered RDS in real-time or substantially real-time to determine respiratory state information indicative of the current position of the user within the larger RC. For example, processing the cleaned RDS using the data processing module 306 can include populating and/or iteratively assessing an array of instantaneous movement and/or positional data, providing a degree of confidence in the momentary respiratory state of the user (e.g., inhalation, exhalation, or paused), or the like. The data processing module 306 can perform data processing including, without limitation, one or more of edge detection, scaling, automatic calibration, or the like.

The respiratory state information can refer to one or more data parameters including, but not limited to, a current location within a respiratory arc, a current respiration direction, a ratio of inhalation to exhalation, a respiration depth, or an average respiratory rate. In some implementations, the filtered data is processed to determine current movement information for the user of the mobile device, such as in addition to or instead of the respiratory state information. For example, the current movement information can refer to rotations of some or all of the body of the user around one or more axes in space.

The device direction data may be analyzed by the data processing module 306 to determine a minimum range of breath (MinRB) value and a maximum range of breath (MaxRB) value for the user of the software application. This information may be updated in real time to dynamically identify the edges of the RC. Operations of an auto-calibration process may draw from previous MinRB and MaxRB values to scale and/or normalize the instantaneous value of the cleaned RDS within a predefined output range (e.g., floating-point values between 0 and 1). Interpolating between the previous and new MinRB and MaxRB values over a given period of time may help to avoid sudden and material changes in the processed RDS, which may help create a subjectively smoother end-user experience.

Given that the device running the software application is constantly adjusting to the breath range of the user, the software application stores some information in device memory about the position of the device (determined using a reference offset corresponding to the body of the user) and can interpolate the current position of the user and a corresponding general range of breath expressed as floating-point values between 0 and 1. This may be referred to as a relative data stream, which constantly adjusts from previous breaths, for example, by shifting based on the speed and depth of user breath. An absolute data stream separately measures and tracks the absolute rotation of the device running the software application relative to the axis of the Earth. The data processing module 306 may use the relative data stream for the auto-calibration process described above.

One full inhalation and exhalation is considered a single RC, which begins at the onset of inhalation, and terminates after exhalation has completed, where a pause may occur at the end of user inhalation or exhalation. Within this technique, these pauses are referred to as the Inhale Pause (IP) and Exhale Pause (EP), respectively. If, during the RC, the RDS indicates an extended EP, a sound-file may be played with verbal output instructing the user to “inhale deeply now.”

The respiratory rate of the user describes the rate at which the user breathes. The average respiratory rate (ARR) may be calculated by counting the number of RCs over a given time period and extrapolating this information to determine how many RCs the user has completed per minute. The equation for calculating ARR is expressed as (# of completed RCs * 60)/window of time (in seconds). With this equation, it is possible to calculate an instantaneous respiratory rate (IRR) after one RC has been completed, the ARR across two or three RCs, or the ARR across an entire session of a given length. In some cases, it may be preferable to provide the user with visual or auditory output related to the ARR rather than the IRR, as the ARR will provide a more stable value when averaged across the last two or three RCs.

The ARR data parameter may be utilized in real-time in various ways. If the ARR for the previous two or three RCs rises well above the ARR calculated across the previous five to ten breaths (appropriate arbitrary values chosen for this example), then a determination can be made that the user may have entered a state of relative hyperventilation. In this case, cues such as verbal audio instruction and/or special tones may be played as output to guide the awareness of the user back to the breath, which may help guide the user toward a slower RR, so as to induce a state of deeper relaxation of the user.

The ARR may be calculated across a small number of breaths and compared to the ARR across a larger number of breaths. Thus, information related to respiratory variance (RV) of the user is acquired over time. Instantaneous respiratory variance (IRV) may be calculated by determining the duration of the two most recently completed RC's (i.e. in milliseconds), comparing the two RCs and determining which was longer, dividing the duration of the shorter RC the duration of the longer, and subtracting the resulting value from one. This value will fall between zero and one, and may be expressed as a percentage. In the instance that the user completes an RC with a duration of two seconds, and another with a duration of four, the resulting equation will be (1- 2/4) which will return a value of 0.5, or 50%. This value may serve as a potential indicator of a distracted mental state, as a high IRV value may indicate that a user is breathing irregularly, and is no longer actively paying attention to their breath. In such cases, the generated soundscape may be adjusted to emphasize sounds that are tightly correlated with the inhalation and exhalation of the user in real time (e.g., melodies derived directly from the contour of the RDS, band-pass filtered noise where the cutoff frequency is controlled directly by the RDS via a transfer function, etc.).

In some implementations, a RV or an IRV of the user may be derived based on a standard deviation calculated across multiple RCs. For example, a running (e.g., windowed) standard deviation may be calculated across a number of RCs. A high standard deviation may indicate a high RV or IRV, whereas a low standard deviation may indicate a low RV or IRV. In some implementations, an ARR or IRR may similarly be derived based on a standard deviation calculated across multiple RCs. The exact methodology for calculating a value (e.g., a RV, an IRV, an ARR, or an IRR) may vary provided a reliable value is consistently calculated across the given implementation.

The ARR and IRV parameters provide information related to the rate and regularity of the RDS as it unfolds over time. Information related to the respiratory depth (RD) is helpful when qualitatively assessing the RC and determining the RDS. Various numbers of terms may be appropriate in describing RD including but not limited to: shallow, deep, small, big, full, and slight. The RD, when considered as a parameter within the evolving RDS, may also be described as the amplitude of the extracted respiratory waveform. Instantaneous Respiratory Depth (IRD) and Average Respiratory Depth (ARD) may also be extracted from the RDS and applied for content selection, parameter mapping, or other output directly to the user via an appropriate modality (e.g., audio or visual). For example, if the IRD falls far below the ARD, a sound file may be played instructing the user to “take a deep breath.”

An example workflow performed at one or both of the data cleaning module 304 or the data processing module 306 will now be described. A sensor (e.g., the sensor 102 shown in FIG. 1) measures and outputs data related to the RC of the user. For example, the data may be used to generate or otherwise derive a portion of a RDS. The rotational position (e.g., the angle of rotation around the x-axis) of the user of the software application may be extracted directly from the sensor at a regular sampling interval. Although one axis of rotation is sampled here, in some implementations, more axes may be used, along with movement or positional data. The measured data parameter is the rate of rotation around along one or more axes, such as the x-axis (e.g., the first derivative of the rotational position). A smoothing filter (e.g., a standard LPF) may be applied to smooth out higher frequency fluctuations in a RDS based on a variable filter-factor. The smoothing filter may be used to remove noise in the rotation rate signal, which can be falsely triggered by erroneous motion. In some cases, the smoothing filter is used to remove noise introduced by the body of the user, for example, the heartbeat of the user.

In some cases, such as where the RDS is derived from the tilt of an accelerometer or gyroscopic sensor of a device running the software application, the workflow may include using directional change operations. One example of a directional change operation or set thereof is defined as follows. A first class is the “Breath Instant,” which stores the instantaneous direction as determined by the smoothed rotation rate around the x-axis. From this value, a direction is calculated as an enumeration including one of three breath directions: “in,” “out,” or “still.” This value is captured at the sampling interval. A second class is the “Breath Moment,” which includes an array of references to a set number of recent instances. The moment analyzes the array of recent instances. After a minimum threshold of “x” consecutive similar instantaneous directions has been encountered, the moment reports the current status (e.g., direction) of the breath. This may, for example, serve as another type of smoothing filter. The number of directionally aligned or otherwise consecutive instantaneous moments that are used for the threshold to be met can be configurable, so as to adjust the direction change sensitivity. Optionally, different sensitivities may be pre-defined for each direction (e.g., for one or both of inhalation or exhalation).

When a direction change is reported by the second class, it may be compared to its previous direction. When a change is detected from one direction to the other, the beginning of a new breath is triggered. The maximum and minimum values (as detected in the RDS) may then be stored as the MinRB and the MaxRB. As a new MaxRB value or a new MinRB value is detected, the processing includes interpolating between the old value and the new value over a short period of time so as not to cause a sudden change in the scaled output value. This interpolation may be achieved via a smoothing filter (e.g., a LPF). The primary output of the workflow is the current rotational position and may be expressed as a percentage of the current breath range.

Another example of such a directional change operation or set thereof may be implemented through determining the instantaneous angular velocity around an axis as calculated at each sampling interval. A variable “counter” may be defined to store a running sum of these instantaneous angular velocities. If the instantaneous velocity is positive (possibly signifying an “in” breath), this running counter will increment, and if the instantaneous velocity is negative (possibly signifying an “out” breath) the counter will decrement.

Numeric thresholds may then be then set to serve as boundaries around the counter value in order to determine the sensitivity of the directional change detector. The “in” breath state, then, will be indicated when the counter crosses the positive threshold, and an “out” breath state is indicated when the counter crosses the negative threshold. Once the counter crosses a threshold in either direction, the runner counter value is reset back to zero, and the new state is sent to the application. One advantage of the latter approach is that it takes into account the velocity of the breath. If the user takes a quick breath, it will trip the boundary immediately, not needing to wait for the minimum amount of time required by a windowed approach.

The parameter extraction module 308 extracts, calculates, and/or otherwise selects low-level data parameters and/or high-level data parameters from the processed RDS. Examples of low-level data parameters include instantaneous position within the RDS, respiratory direction, the onset time of a new RC, the average respiratory rate across multiple RCs, and the percentage of variance in breath length and depth across multiple RCs. The parameter extraction module 308 uses definitions of the parameters to extract, calculate, or otherwise select the low-level and high-level data parameters. In some implementations, the definitions of the parameters used by the parameter extraction module 308 may be configurable. For example, the application may include functionality for allowing a user thereof to select the definitions, such as from a list of available parameter definitions.

Performing the parameter extraction may include extracting, identifying, calculating, or otherwise determining one or more parameters from the respiratory state information based on definitions of one or more parameters. For example, the parameters can be extracted from one or more of an ARR, an IRR, a RRD, an IRV, an IRD, or an ARD of the user. In implementations in which the filtered data is processed to determine current movement information for the user of the mobile device (e.g., in addition to or instead of the respiratory state information), the parameter extraction may also be performed against the current movement information. In some implementations, the definitions of selection criteria used for extracting the parameters from the respiratory state information (and, as applicable, from the current movement information) may be configurable.

As used herein, a parameter may be or refer to a configuration or setting value which may be used to change, control, or otherwise cause some audio and/or visual output to a user of a software application. In some implementations, the parameters may be audio parameters. The audio parameters correspond to one or more of a volume of an audio channel, a pitch of a synthesized tone, a playback speed of an audio file, a cutoff frequency for a filter, or an audio effect. In some implementations, the parameters may be visual parameters. The visual parameters correspond to changes in one or more GUIs of the application. In some implementations, the parameters may be both audio parameters and visual parameters.

In some cases, these-lower level data parameters may be assessed and synthesized to extract the higher-level data parameters, including the establishment of various “user states.” One example of a user state may be “distracted,” which may represent, indicate, or otherwise correspond to a respiratory rate, respiratory variance, and instantaneous respiration speed (or a range thereof) used to determine that the respiratory behavior of the user has drastically changed. User states as used herein may refer to an emotional, mental, and/or physiological state of the user of the software application. The user states may be derived to better understand how the RDS should be processed, such as by the mapping of extracted parameters.

The parameter mapping module 310 maps the parameters (which may, for example, include, but are not limited to, playback speed, filter cutoff frequency, mix ratios, triggering of audio file playback, and the like) to aspects of an audio signal which is, has been, or will be output for perception by the user. In particular, performing the parameter mapping includes mapping the parameters extracted from the filtered data of the user onto parameters for digital signal processing. For example, one or more of the audio parameters can be mapped to music, speech, or other audio. In some implementations, one or more of the audio parameters may also or instead be mapped to visual content, such as which may be output for display at a display of the device. The mapping can include generating a data representation using a transfer function. In some implementations, the transfer function may be continuous. In some implementations, the transfer function may be non-continuous. In some implementations, the transfer function may be linear. In some implementations, the transfer function may be non-linear.

The signal production module 312 produces an output signal indicating the mapped audio parameters and outputs the output signal, such as to an output component of the device (e.g., the output component 106 shown in FIG. 1). The output signal may include audio and/or visual data perceptible to the user so as to affect a respiratory pattern of the user. The respiratory pattern of the user is subsequently modified based on such output from the signal production module 312. Data representative of the modified respiratory pattern can be fed back into the data acquisition module 302.

The processing of user breath information via a RDS as disclosed herein relates to the processing of data points of the RDS, of which a current data point of the RDS being processed at a given time is referred to as a current data point. In some implementations, the relative data stream, described above, may use a reference data point which is half a RC behind the current data point. In such an implementation, processing performed with respect to the breath of the user may focus on the reference data point as a measure of the breath activity of the user a short time (e.g., one second or less) before adjustments to content presented to the user based on respiratory biofeedback are made. In some implementations, reference data points throughout some or all of the relative data stream (e.g., one or more respiratory cycles thereof) may be averaged to evaluate user breath information. In some implementations, each current data point may be instantaneously processed to evaluate user breath information, so as to continuously update the user breath information which each new data point that is identified and processed.

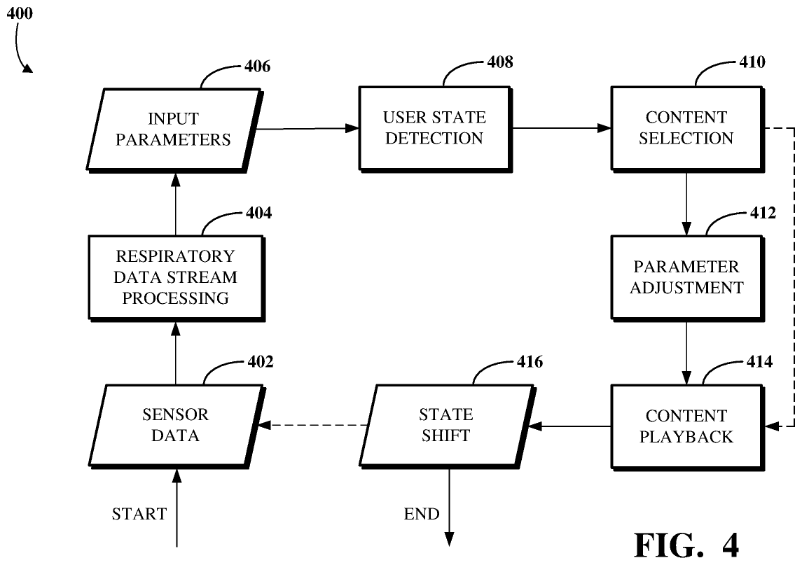

FIG. 4 shows a block diagram of an example workflow 400 for respiratory biofeedback-based content selection and playback. The workflow 400 represents a biofeedback cycle which uses software modules, including one or more of the software modules shown in FIG. 3, and output of those software modules to determine how to adjust output provided to a user of a software application based on the respiratory biofeedback information of the user. In particular, the workflow 400 may be continuously repeated during a session run via the software application. The session may, for example, be a guided session or a freestyle session (e.g., where the user is acting on their own without guidance).

The workflow 400 begins with sensor data 402, which represents data produced using one or more sensors of a device, such as the device 100 shown in FIG. 1. For example, the sensor data 402 may be or refer to data produced using an accelerometer, a gyroscope, or another sensor of a mobile device, such as a smartphone. The sensor data 402 is preferably sensor data which has undergone processing at one or more software modules to prepare the sensor data for further processing in extracting and mapping parameters. For example, the sensor data 402 may be sensor data which was acquired using the data acquisition module 302 shown in FIG. 3 and processed at the data cleaning module 304 shown in FIG. 3. Alternatively, the sensor data 402 may be raw sensor data such as in the form originally produced by the one or more sensors. The sensor data 402 may be understood to include data for a given time window, which may be a defined or discrete time interval or a configurable unit of time.

The sensor data 402 is processed at a RDS processing module 404 to identify one or more respiratory biofeedback qualities of the sensor data 402. In particular, the RDS processing module 404 uses the sensor data to determine a respiratory curve, a respiratory stability, and a respiratory rate. The respiratory curve represents the relationship between inhalation and exhalation in the user including the data points representing a first curve between a time before a breath is taken and a time at which the user finishes inhaling and data points representing a second curve between the time at which the user finishes inhaling and a time at which the user finishes exhaling. In at least some cases, the respiratory curve may be derived based on an orientation of the device on the user, such as by measuring rotation to a peak rotational distance from an origin position of the device.

The respiratory stability is a measure of the variance of respiration in lungs of the user used to determine whether the user is breathing at a regular pace. Respiratory stability may be defined as the inverse of RV, such that, for example, a low RV may indicate a high respiratory stability. Respiratory stability may be modeled after healthy or otherwise typical respiratory curves and the correlations thereof to different emotional states. In particular, respiratory stability is a function of emotional state in that the emotional state of the user may bring about a change in the amount of respiratory stability in the user's breath notwithstanding possible inconsistencies therein. For example, if the user of the software application takes six breaths at a regular pace and then two more breaths at a very quick pace, and thereafter a few more breaths at a very slow pace, the measurement of respiratory stability indicates that the user is irregularly breathing. This may infer a more generally distracted than relaxed state in the user. This information is useful to extract parameters from the user's RDS and further to understand how to map those parameters to cause a desirable change in the user's respiratory biofeedback loop. Respiratory stability may generally be measured using a standard deviation from an average resting breathing rate. For example, where a user over the course of five breaths has a relatively small deviation in terms of the length of their breaths, an inference can be made that the user has a relatively high respiratory stability as compared with a different user who over the course of five breaths has a relatively larger deviation.

The output of the RDS processing module 404 is represented as input parameters 406, which are or refer to measured values in one or more of the respiratory curve, respiratory stability, or respiratory rate of the user, and which may in at least some cases relate to waveform representations of such values. The input parameters 406 are received at and processed by a user state detection module 408 to infer a user state of the user of the software application. The user state refers to an emotional, mental, and/or physiological state of the user inferred based on the RDS of the user. The user state detection module 408 generally performs some classification against the input parameters 406 to derive the user state of the user.

Various user states may be modeled empirically such as by the processing and analysis of sets of user RDS data collected from one or more users of the software application (e.g., from the same device or from different devices). In at least some such cases, the users may be asked to verify their own user states in order to accurately label RDS data into a particular user state group. For example, before a guided session begins, after a guided session ends, and/or near the beginning or end of a guided session, the software application may ask a user to indicate his or her emotional, mental, and/or physiological state. Over time, the software application correlates certain respiratory measurements with certain states and becomes able to intelligently infer a user state based on the input parameters 406. In some implementations, the software application may leverage a machine learning model to statistically analyze sets of RDS data and label same into known user states. In some implementations, user state modeling may be performed external to the software application. In some such implementations, indications of a correspondence between one or more such user states and human respiratory information may be derived from a third party resource.

Referring by example to emotional state detection, the user state detection module 408 may process the input parameters 406 to infer an emotional state of the user of the software application. This emotional state information will be useful later in the workflow 400, so as to better understand how to adjust aspects of a session performed via the software application to achieve a desired objective for the user. For example, where the input parameters 406 indicate that the user took a large breath followed by several short breaths, such as which may be illustrated by the respiratory curve thereof, the user state detection module 408 may infer the user to be in a sad emotional state—this is because these short breaths may be correlated with sobbing. In another example, where the input parameters 406 indicate that each breath represented by the input parameters 406 is consistent in length and depth, this may instead infer the user to be in a happy, calm, or otherwise relaxed emotional state.

The user state inferred using the user state detection module 408, as described above, is used in the workflow 400 to determine whether and how to adjust content for playback to a user of the software application so as to hopefully cause the user to achieve a desired objective associated with a current session. For example, if the user is in a meditation guided session and his or her user state is inferred to be sad, the software application, via the workflow 400, may use that information to adjust content output to the user to help trigger a happier or calmer reaction by the user, so as to help the user arrive at a more relaxed emotional state as is the objective of the meditation guided session.

Thus, a content selection module 410 receives an indication of the user state inferred using the user state detection module 408 and selects content for playback to the user based thereon and based on the particular session performed by the user. The content selected using the content selection module 410 corresponds to cues for guiding the user through a guided session and may be or refer to audio and/or visual content to be presented to the user via one or more output components of the device running the software application, for example, the output component 106 shown in FIG. 1. The content may be pre-existing content generated before the session began, such as which may be accessible within a data store associated with the software application. Alternatively, the content may be generated during the session, such as in response to various RDS measurements and/or other events in a session.

The content may generally be separated into two groups, including a first group for body-focused content and a second group for mind-focused content. Body-focused content may, for example, include or refer to content related to progressive relaxation, body scanning, and breath focus. Mind-focused content may, for example, include or refer to content related to gratitude, encouragement, acceptance, positive-visioning, loving kindness, self-compassion, and intention setting. Each of the body-focused content and the mind-focused content may include audio content and/or visual content.

Audio content selected using the content selection module 410 may include or refer to particular tones which may be layered on top of an already playing audio track (e.g., a musical scale, chord, or individual note), a change in an already playing audio track (e.g., adjustments to the volume, the tempo, or another aspect thereof; filtering; replacement of the audio track with a new audio track, such as by the gradual phasing out of the current audio track; a change in musical scale or chord, such as from a minor to major or from a major to minor; or another change), spoken cues which may be layered on top of an already playing audio track, or other audio content. Particularly, the spoken cues may include guidance related to the session being performed by the user and/or generally encouraging commentary intended to stimulate a positive change in the user state of the user toward an objective of the session.

Visual content selected using the content selection module 410 may include or refer to particular images or video frames (e.g., of singular image, animation, or video content) being displayed at the device running the software application, changes to an existing image or video being displayed at the device (e.g., adjustments to the color, brightness, contrast, or another aspect thereof), visual cues which may be layered on top of an already displayed image or video, or other content. Particularly, the visual cues may include text or pictorial guidance related to the session being performed by the user and/or generally encouraging text or imagery intended to stimulate a positive change in the user state of the user toward an objective of the session.

In some implementations, some or all of the types of content which can be selected using the content selection module 410 may be selectively configured by the user of the software application. For example, a GUI of the software application may enable a user thereof to selectively enable or disable certain types of content, for example, content including chords, bells, wind chimes, strings, water, voices, wind, or the like. In another example, the same GUI or another GUI may enable the user to selectively control a value range for a given type of content, so as to vary the volume, frequency of presence or use, or other qualities of the content. In some such implementations, the content selection module 410 will reference these user configurations when determining the content to select.

The content selection module 410 uses the user state derived using the user state detection module 408 to select content based on an understanding of the intended objective for the session performed by the user and based on the current user state. For example, where the session is a sleep induction guided session and the user state is awake, angry, or another state different from one commonly understood to be associated with relaxation, the content selection module 410 may select audio and/or visual content intended to cause the user to become more relaxed. Examples of such content may include, but are not limited to, audio content in the form of a gentle rainfall or waves gently meeting the seashore, or visual content in the form of an image of nature. To the extent such audio and/or visual content is already being presented to the user, the content selection performed using the content selection module 410 may instead refer to an adjustment to such existing content, such as to the volume or tempo of the audio content and/or to the brightness or of the visual content. Given the potentially very large number of combinations of session and user state, there may be a very large number of different possible audio and/or visual content selections made using the content selection module 410.

In some implementations, the user of the software application may further select an additional layer of guidance for use in a session. In such a case, the content selection module 410 may further use that indication to select content for playback. For example, as a library of pre-recorded content may include multiple modularized cues, the content selection module 410 may use weights assigned to the different labels or categories of content to dictate the extent to which each such label or category is highlighted over the course of the session. For example, the user may decide to emphasize “gratitude,” with some included elements of “relaxation” and “self-compassion.” In such a case, the content selection module 410 may weight pre-recorded content having corresponding labels more heavily when determining the content to select for playback.

The timing of content to be played back to the user after selection using the content selection module 410 is to be considered. For example, to create musical or other audio content that extend to a given length based on input from the RDS, different versions of pre-recorded audio files may be created that can each seamlessly loop, such that the user is unable to detect where a recording begins and ends. This may be accomplished by splicing and looping at a specific location in the audio, for example, at a specific location between performed notes. Alternatively, a seamless loop from a single held note or chord may instead be used. It is possible that the playback speed of audio files that are seamless looped in this way may be adjusted to create new harmonies that can be sustained for a desired duration. Additionally, recorded meditations may be modularized by segmenting the recorded audio into predefined regions, which may be called up and played back to the user in a way that may be randomized or semi-structured.

Depending on the content selected using the content selection module 410, a parameter adjustment module 412 may be used to adjust one or more parameters of the content presented to the user. For example, where the content selection module 410 determines to adjust audio and/or visual content already being output to the user, the parameter adjustment module 412 may be used to adjust such content accordingly. The parameter adjustment module 412 in particular receives an indication of the adjustment or adjustments to be made from the content selection module 410 and makes such adjustment or adjustments. For example, the parameter adjustment module 412 may effect a change in volume of an audio track being played or may effect a change in brightness for visual content being displayed. In some implementations, such as where the content selected using the content selection module 410 is or refers to new audio content and/or new visual content to present to the user and not simply to changes to make to audio content and/or visual content already being presented to the user, the workflow 400 may skip the parameter adjustment module 412.

A content playback module 414 causes the outputting of the content as selected using the content selection module 410, and, as the case may be, adjusted using the parameter adjustment module 412, to the user of the software application by causing the outputting to one or more corresponding output components of the device running the software application. For example, the content output to the user may, but not necessarily, be specifically timed to correlate with events identified within the processed RDS, including the onset of a new RC. In this way, the playback of selected content may be triggered directly by the inhalation or exhalation of the user, and the pacing of a session may be adjusted by adjusting the number of breaths that occurs between each new auditory prompt. In some cases, this value may also be randomized to “humanize” the experience and/or create some level of unpredictability.

After content is presented using the content playback module 414, a state shift 416 is detected to indicate the progression of the user toward a given objective of the session being performed thereby, such as based on the content output using the content playback module 414. For example, the state shift may be detected by the further collection and processing of new sensor data, so as to infer a new user state and determine whether that new user state is different from the user state inferred using the user state detection module 408. Where the session is ending or has ended, the workflow 400 may terminate after the state shift 416, or, in some implementations, prior to the state shift 416. Where the session is not ending, the workflow 400 may repeat by the collection and processing of a new set of sensor data.

In some implementations, data may be collected from a user of the software application in real time as part of the workflow 400 in order to learn how different types of content affect the user over time. For example, a person other than the user who is leading a guided session, either live or via pre-recorded cues, may provide a variety of content for the guided session. Different content thereof may be selected for playback to the user at different times, such as based on inferred user states of the user, to steer the guided session and the participation of the user therein. In some such implementations, the learned output may be used to derive custom experiences for a given user of the software application, such as by the customized creation of audio and/or visual content which is learned to more effectively guide the given user to the desired objective of a given guided session.

In some implementations, biometric user data received from a secondary device may be processed along with the sensor data 402 as part of the workflow 400. For example, the user state detection module 408 may be configured to derive a user state based on both the input parameters 406 and also based on such other biometric user data, for example, heart rate data for the user as may be sensed using a secondary device in communication with the device running the software application. In some implementations, secondary respiratory information may be collected from the secondary device, such as which may itself be capable of being processed as a RDS. In some such implementations, a sensor fusion scheme can be used to combine the respiratory information collected at each of the secondary device and the device running the software application. For example, the sensor fusion scheme may, in at least some cases, improve the accuracy of the RDS capture processing.

In some implementations, a scoring system may be used in connection with the workflow 400, for example, to measure user participation within a guided session. For example, the scoring system can be used to measure a respiratory or other biometric (e.g. heart rate variability) value achieved at the end of a guided session. In another example, the scoring system can be used to measure a value indicating how well the user tracked with the guided session or how quickly the user achieved the objective of the guided session (e.g., falling asleep, where the guided session is a sleep induction guided session). The scores measured for the user may be tracked over time to show user progress toward the objective of a given guided session.

In some implementations, a content or session recommendation system may be used in connection with the workflow 400, for example, to recommend certain types of content and/or certain types of session for the user. For example, recommendations can be presented to the user based on previous respiratory biofeedback data collected for the user (e.g., from past sessions). In another example, recommendations can be presented to the user based on a score presented by a scoring system, such as in response to the performance of a session by the user. In either case, the recommendation may indicate tips or suggestions for the user to improve his or her breathing activity or technique, such as by recommending changes to amounts of exercise performed by the user, recommending changes to the length of time a user inhales or exhales, recommending that the user participate in a certain type of guided session, or the like.

In some implementations, a genetic algorithm or other algorithm or technique beyond the approach described above with respect to the workflow 400 may be used to select and/or adjust content as part of the workflow 400. For example, a genetic algorithm may be used to evaluate content available for selection or adjustment according to learned breath activity of the user of the software application.

In some implementations, a wearable device application running on a wearable device may be used in connection with the workflow 400 to improve breathing activity of the user of the software application running on the mobile device. The wearable device application may be configured to present output to the user of the software application, in which the output is intended to cause the user to breath in a certain manner. For example, the output may be vibrations presented in discrete time intervals to cause the user to achieve a desired breath rhythm, which is a controlled pattern for the user to reference in connection with his or her breathing while using the software application.

In some such implementations, a breath score or other score as may be calculated using the software application may be determined based on how well the user matched the breath rhythm output by the wearable device. For example, the software application can keep track of the times at which output is presented by the wearable device application and of the times at which the user inhales and/or exhales. Those times can be compared to determine how well the user maintained the breath rhythm.

In some such implementations, an emotional state of the user may be detected by extracting and modeling parameters obtained using one or more sensors of the wearable device. For example, a heart rate or heart rate variance of the user can be obtained at the wearable device while the user participates in a session through the mobile device running the software application. The heart rate or heart rate variance may be used to determine an emotional state of the user, for example, as elsewhere described herein based on models of emotional states using breath information. In some implementations, the emotional states determined using the parameters obtained by the one or more sensors of the wearable device may be compared against emotion states determined using RDS information.

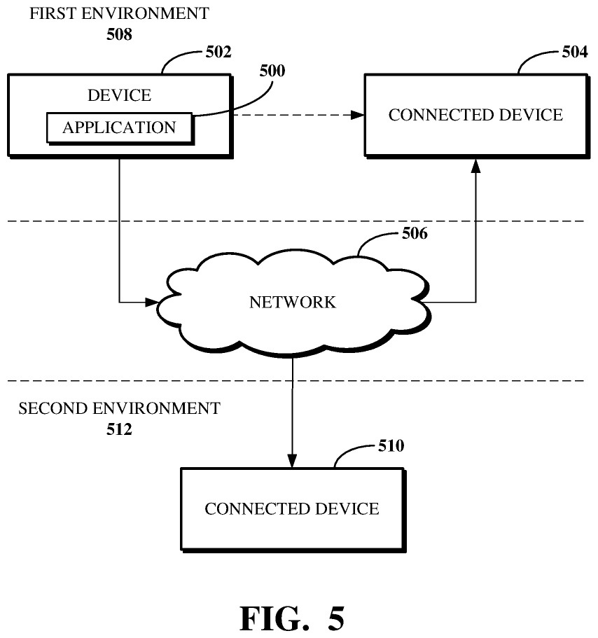

FIG. 5 shows a block diagram an example of device adjustment of a connected device using a software application 500 for respiratory biofeedback-based content selection and playback, which may, for example, be the software application 200 shown in FIG. 2. The software application is run on a device 502, which may, for example, be the device 100 shown in FIG. 1. The software application 500, through the device 502, is able to communicate with a connected device 504 over a network 506, which may, for example, be a local area network, a wide area network, a machine-to-machine network, a virtual private network, or another public or private network. The communication between two or more devices over the network 506 may use one or more network protocols, such as using Ethernet, TCP, IP, power line communication, Wi-Fi, GPRS, GSM, CDMA, Z-Wave, ZigBee, another protocol, or a combination thereof.

The connected device 504 is a network-connected computing device or device with some form of Internet-of-Things (IoT) functionality which may be operated over the network 506. Examples of what the connected device 504 may be include, but are not limited to, a smart lightbulb, a smart light switch, a smart thermostat, a haptic mat or table, a vibrotactile mat or table, or a wearable device including, but not limited to, a smart watch. The particular functionality of the connected device 504 is based on the particular kind of device it is, but in any event some or all of such functionality of the connected device 504 may be triggered (e.g., selectively operated) using signals transmitted from the software application 500 via the device 502.

In particular, functionality of the connected device 504 may be triggered based on the RDS of the user of the software application 500. For example, functionality of the connected device 504 may be triggered upon the identification of a breath event, which may, for example, be or include the user beginning to inhale, the user beginning to exhale, the user holding his or her breath, the user achieving a certain respiratory rate or respiratory stability, or another breath event. In another example, functionality of the connected device 504 may be triggered upon the derivation of a user state based on the RDS of the user, such as based on a determination that the user is or is not in a relaxed state.

In yet another example, functionality of the connected device 504 may be triggered upon the user reaching a transition point within a session. For example, as described above, a guided session may in some cases include one or more transition points defined or reached by the user meeting some breath event or user state event, which transition points may be based on the particular objective of the guided session. For example, in a sleep induction guided session, a transition point may mark the point at which the user falls asleep. In another example, in a meditation guided session, a transition point may mark the point at which the user's respiratory rate and respiratory stability achieve specified values. In still a further example, functionality of the connected device 504 may be triggered upon a determination that the user, based on his or her breath activity, is experiencing breathing issues, for example, upon a determination based on the respiratory rate and respiratory curve of the user indicating that he or she is hyperventilating.

The particular functionality triggered in the connected device 504 depends upon the triggering event and the session. For example, the middle of a meditation session, indicated by a transition point reached by the respiratory rate and respiratory stability of the user achieving specified values, may trigger a smart lightbulb to dim to a lowered brightness level, and the end of the meditation session, indicated by a different transition point, may trigger that smart lightbulb to return to the original brightness level. In another example, the haptic or vibrotactile functionality of a mat, table, or wearable device (e.g., a smart watch) may be triggered in a different type of guided session, such as upon the user reaching a transition point therein.

As described herein, the achievement by a user of the software application 500 of reaching a given transition point within a session may be inferred in one or more ways. For example, a transition point achieved by the user being in a certain user state may be inferred by modeling user states based on different respiratory information with a standard deviation and potentially a threshold value for respiratory rate and respiratory stability, so as to prevent false positive events from causing a transition, In another example, biofeedback markers may be used to infer the achievement by the user of reaching a transition point, such as where the transition point is defined to occur where a certain breath event indicated by the RDS occurs.

As shown, the connected device 504 is located within a first environment 508, which is the same environment in which the device 502, and hence the user of the software application 500, is located. Thus, although communication with the connected device 504 is described herein as being through the network 506, in some implementations, communications with the connected device 504 may instead be made directly, for example, over Bluetooth®, infrared, or another direct connection protocol.

Furthermore, in some implementations, the software application 500, via the device 502, may be used to trigger functionality of a connected device 510 located in a second environment 512 different from the first environment 508. For example, the second environment 512 may be a room outside a room in which the user of the software application 500 is located, a building separate from a building within which the user is located, or even a city, state, or country separate from that in which the user is located. In some such implementations, the triggering of functionality of the connected device 510 within the second environment 512 may be performed to signal a change in user state or in user breath activity to a person within the second environment 512. The use of the connected device 510 at the second environment 512 may thus provide real-time or close to real-time updates related to the biofeedback information of the user of the software application 500 to a person located within that second environment 512.

For example, in a healthcare setting, a user of the software application 500 may be a patient in a clinic or hospital. The software application 500 can trigger functionality of the connected device 510 to signal, such as by a flashing light or other means, to a healthcare provider that the patient has achieved a certain user state or that certain breath activity of the user is occurring. In another example, in a transportation setting, a user of the software application 500 may be a passenger on board an airplane in flight. The software application 500 can trigger functionality of the connected device 510 to signal, such as by a flashing light or other means, to a flight attendant that the user is experiencing breathing issues, such as which may be inferred by the RDS of the user. In yet another example, in a health and fitness setting, such as a yoga or like studio, a user of the software application 500 may be an exercise or activity participant. The software application 500 can trigger functionality of the connected device 510 to signal, such as by a flashing light or other means, to a person leading the exercise or activity that the user has completed some portion thereof or otherwise has achieved a certain user state or breath event.

In some implementations, the use of a connected device, such as the connected device 504 or the connected device 510, may be in connection with a guided session, as described herein. Thus, certain actions or events inferred to occur based on the RDS of the user of the software application 500 may be used by the software application 500 to trigger functionality of a connected device.

In some implementations, a connected device, such as the connected device 504 or the connected device 510, may be other than a “smart” or IoT device. For example, the connected device may be a musical instrument digital interface (MIDI) controller which receives control data from the software application and for which functionality is configured or otherwise operated using that control data. For example, a user of the software application may configure a MIDI controller to receive commands upon the occurrence of certain events during a session performed using the software application, such as at a time the user's breath is detected, after the user is determined to not have taken a breath for a certain amount of time, upon the transition of one piece of audio and/or visual content to another, or the like. The MIDI controller may use the commands received from the software application to generate audio content in the form of music, as may be configured at or otherwise using the MIDI controller. For example, the relative breath wave of the user of the software application 500 may be expressed in a RDS as a MIDI value to enable MIDI control based on the RDS or the processing thereof.

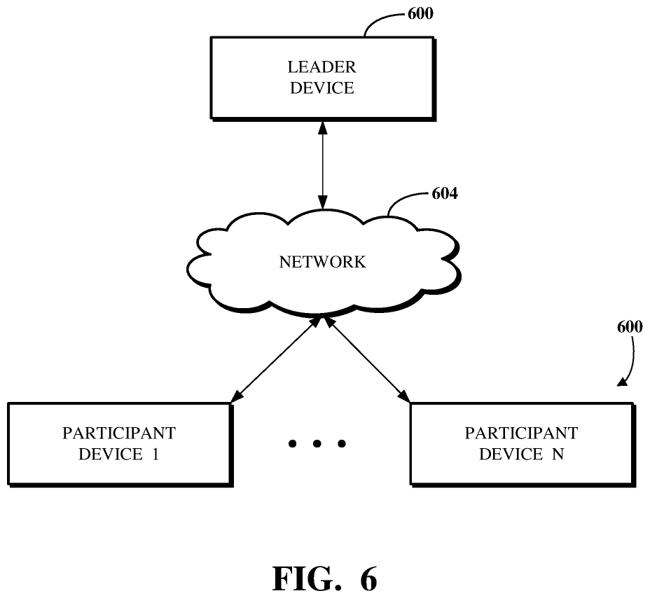

FIG. 6 shows a block diagram of an example of a multi-user system for respiratory biofeedback-based content selection and playback. As shown, the multi-user system includes a leader device 600 and one or more participant devices 602, shown as participant device 1 through N, in communication with the leader device 600 over a network 604, which may, for example, be the network 506 or a similar network. The leader device 600 may be considered to be used by a user who is leading some session, such as a guided session, in real-time, and the participant devices 602 may be considered to be used by separate users who are participating in that session lead by the user of the leader device 600. For example, the multi-user system of FIG. 6 may represent an approach for a virtual meditation or other session, such as which may be performed remotely by some or all participants thereof.

Using this multi-user system, a guided session may be led by a single user and participated in by multiple other uses of a software application, which may, for example, be the software application 200 shown in FIG. 2. A single, multi-tenant instance of the software application 200 may be operated, such as which may be served and streamed from the leader device 600 or a separate server device (not shown), which may, for example, run a web server to which multi-user access is enabled for the single instantiation of the software application. This single, multi-tenant instance approach enables the leader and participants in the guided session to share their biofeedback information, and, thus, breath events and other occurrences as may be inferred from each user's RDS, in real-time with one another. In some implementations, however, multiple, single-tenant instances of the software application 200 may be operated, in which case a separate layer of reporting biofeedback information between instances is used.

Regardless of the particular approach, a global synchronization mechanism may be used to synchronize user activity, such as within the multi-user system of FIG. 6. The global synchronization mechanism enables each user of the software application to follow their specific breath information to a waveform (e.g., a sinusoidal waveform) generated by a local device clock of the device they use to run the software application. Provided that the various devices connected to a multi-user guided session are connected to a network having a clock controlled by a geolocation service, such as GPS, timing of user activities can be synchronized accordingly. In some implementations, the global synchronization mechanism may indicate a number of participants to a session, or a number of participants who also happen to be concurrently using the software application running on their own devices even if not part of a same session, for display to the user of the software application.

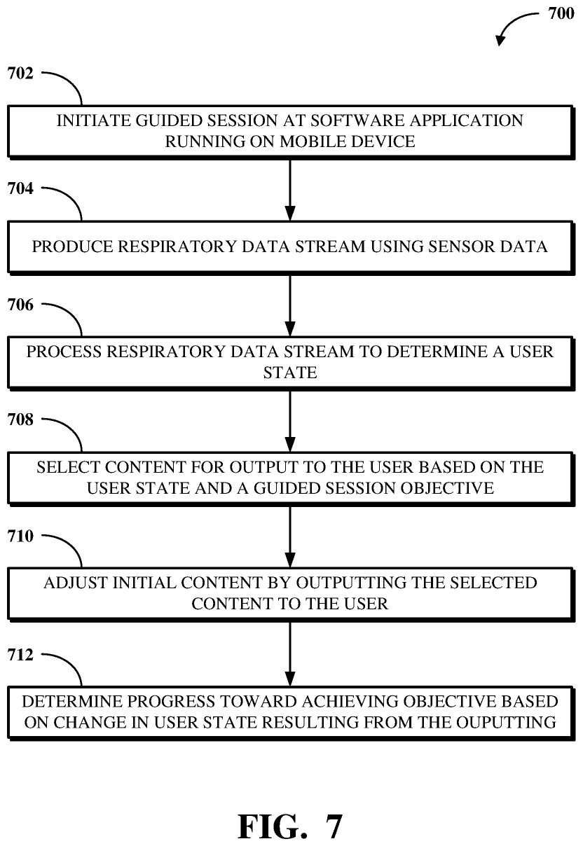

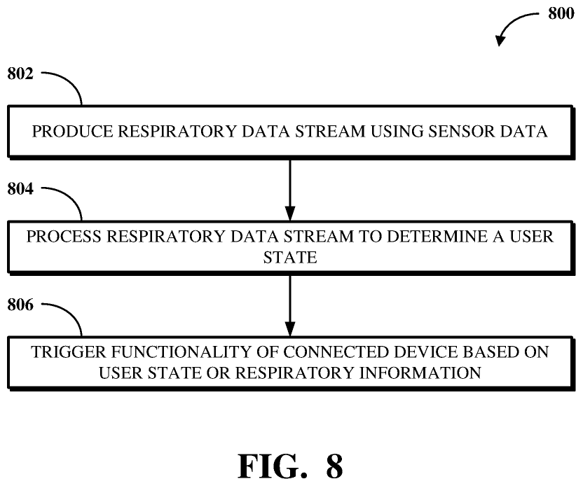

To further describe some implementations in greater detail, reference is next made to examples of techniques which may be performed in connection with systems for respiratory biofeedback-based content selection and playback, such as for guided sessions and device adjustments. FIG. 7 shows a flowchart showing an example of a technique 700 for respiratory biofeedback-based content selection and playback for a guided session. FIG. 8 shows a flowchart showing an example of a technique 800 for respiratory biofeedback-based content selection and playback for a device adjustment.

The technique 700 and/or the technique 800 can be executed using computing devices, such as the systems, hardware, and software described with respect to FIGS. 1-6. The technique 700 and/or the technique 800 can be performed, for example, by executing a machine-readable program or other computer-executable instructions, such as routines, instructions, programs, or other code. The steps, or operations, of the technique 700 and/or the technique 800, or another technique, method, process, or algorithm described in connection with the implementations disclosed herein can be implemented directly in hardware, firmware, software executed by hardware, circuitry, or a combination thereof.

For simplicity of explanation, the technique 700 and the technique 800 are each depicted and described herein as a series of steps or operations. However, the steps or operations in accordance with this disclosure can occur in various orders and/or concurrently. Additionally, other steps or operations not presented and described herein may be used. Furthermore, not all illustrated steps or operations may be required to implement a technique in accordance with the disclosed subject matter.

Referring first to FIG. 7, the technique 700 for respiratory biofeedback-based content selection and playback for a guided session is shown. At 702, a guided session is initiated at a software application running on a mobile device of a user. The guided session may be initiated by the user of the software application selecting the guided session within the software application. In some implementations, the selection to initiate the guided session may include a user selection of an additional layer of guidance used to enhance the guided session.

At 704, a RDS is processed using data obtained from one or more sensors of the mobile device. The RDS represents a stream of respiratory information of the user of the software application while the mobile device rests on the user. The RDS may, for example, include a respiratory curve, a respiratory stability, and a respiratory rate of the user. In some implementations, producing the RDS may include denoising the data obtained using the one or more sensors using a motion noise baseline determined for an environment in which the user is located during performance of the guided session.

At 706, the RDS is processed to determine a user state of the user of the software application. Processing the respiratory data stream to determine the user state of the user may include classifying one or more of the respiratory curve, the respiratory stability, or the respiratory rate according to user state models to infer the user state, which user state may be an emotional state, a mental state, a physiological state, or a combination thereof. In some implementations, the user state may be determined using the RDS and using biometric user data received from a secondary device in communication with the mobile device.

At 708, content for output to the user is selected based on the user state and based on a defined respiratory objective of the guided session. The content may be audio content and/or visual content. The content is selected with the goal of enabling the user of the software application to achieve the defined respiratory objective of the guided session, which may be specific to the guided session. For example, the defined respiratory objective of a sleep induction guided session may be achieving a respiratory curve, stability, and/or rate consistent with those understood to indicate a state of sleep. In another example, the defined respiratory objective of a stress or relaxation management guided session may be achieving a respiratory curve, stability, and/or rate associated with a low heart rate.

The selected content may refer to new content to replace the initial content or an indication of parameters of the initial content to be adjusted. For example, selecting the content for output to the user based on the user state and based on the defined objective of the guided session may include determining, based on at least one of the user state or the defined objective of the guided session, to adjust one or more parameters associated with initial content. For example, where the initial content includes music output to a speaker (e.g., of the mobile device or another device), the one or more parameters associated with the initial content may correspond to one or both of a volume or tempo of the music. In another example, selecting the content for output to the user based on the user state and based on the defined objective of the guided session may include selecting new content to use to replace the initial content or to be played on top of the initial content.

At 710, the initial content previously output to the user during the guided session is adjusted using the selected content, and, specifically, by outputting the selected content to the user. The particular manner in which the initial content is adjusted is dependent upon the particular type of the selected content. For example, where the selected content is or includes parameters to use to adjust aspects of the initial content, the initial content remains in playback while those aspects thereof are adjusted. In another example, where the selected content is or includes new content for playback on top of or replacing the initial content, adjustment is made accordingly.

At 712, a progress of the user toward achieving the defined respiratory objective of the guided session is determined based on a change in the user state resulting from the outputting of the selected content. A biofeedback loop including processing respiratory data streams of the user, selecting new content for output to the user based on the respiratory data streams, and outputting the new content to the user is repeated until the guided session is completed.

In some implementations, a determination may be made, based on the respiratory information of the respiratory data stream or the change in the user state, that a transition point of the guided session has been reached by the user. For example, a transition point may indicate a transition from one level or state to another. In such a case, an aspect of the guided session may be adjusted as a result of the user reaching the transition point of the guided session. For example, a first transition point in a sleep induction guided session may be reached by the respiratory information of the user indicating that the user has a respiratory curve, stability, and/or rate consistent with that of someone expected to shortly fall asleep, such that they are in a first consciousness level. A second transition point in that sleep induction guided session may be reached by the user achieving a second consciousness level. This may also be the case in other types of guided session, including, without limitation, meditation guided sessions. Further respiratory data streams of the user of the software application may continue to be produced and used to output content for presentation to the user until the software application determines the user has fallen asleep or achieved a deep state of meditation (i.e., a deep state of consciousness).

In some implementations, responsive to a completion of the guided session, determining a score for the user based on progress by the user toward achieving the defined respiratory objective of the guided session.

In some implementations, the technique 700 may be repeated with other RDS data. For example, a second RDS of the user may be produced after the content is adjusted at 710. The second RDS may be a RDS separate from the RDS which was ultimately used to adjust the content at 710. Alternatively, the second RDS may refer to a different segment or other part of the same RDS which was ultimately used to adjust the content at 710. The second RDS may be processed to select second content to output to the user, such as based on the guided session, which second content may then be output to the user and configured to change the user state of the user from a first state to a second state.

In some implementations, the technique 700 may further include transmitting, to a connected device over a network, a command configured to trigger functionality of the connected device based on the user reaching the transition point of the guided session. In some such implementations, the connected device may be located at a second environment different from an environment in which the user is located during performance of the guided session. In some such implementations, different functionality can be triggered at different times during a guided session. For example, based on a first consciousness level of the user of the software application, a first command may be transmitted to a smart light to cause the smart light to decrease a brightness setting of the smart light to a first level. Thereafter, based on a second consciousness level of the user of the software application, a second command may be transmitted to the smart light to cause the smart light to decrease the brightness setting of the smart light to a second level.

In some implementations, the technique 700 includes outputting feedback at a wearable device worn by a user of the software application while a session is in progress. A wearable device application running at the wearable device may be in communication with the software application while the mobile device running the software application rests on or otherwise against the user during the session. For example, the software application may communicate respiratory information determined for the user during the session to the wearable device application to cause certain types of output at the wearable device during the session. In another example, the wearable device application may be configured to output tactile feedback (e.g., using a haptic sensor of the wearable device) to indicate a breathing rhythm for the user to achieve during the session.

The breathing rhythm may be configured at the mobile device and/or at the wearable device. The breathing rhythm indicates times at which to inhale and/or times at which to exhale. For example, the breathing rhythm may be expressed as a series of vibrations output at discrete time intervals. In some cases, the breathing rhythm may be based on the particular type of guided session and/or based on a current portion of a guided session (e.g., whether the user has passed one or more transition points).

In particular, the technique 700 may include configuring a wearable device application to output feedback intended to cause the user of the software application to breath in a particular manner and/or at particular times during the course of a session. The wearable device application may be directly configured manually by the user, indirectly configured manually by the user (e.g., through the user entering configurations in the software applications in which those configurations are then transmitted to the wearable device application), configured by the software application based on a type of guided session, or otherwise configured.

The session is initiated, and, during the session, output is presented to the user via the wearable device according to the configurations. For example, the configurations may cause a vibration of the wearable device against an arm, leg, or other part of the user at discrete time intervals. This may have the benefit of causing the user to breath in sync with those vibrations. Breathing in a controlled rhythm may improve the respiratory activity of the user, thereby improving a respiratory rate and respiratory stability of the user. This, in turn, may cause a more accurate parameter mapping, for example, by using a more consistent stream of respiratory information to affect the changes in outputs presented to the user as part of the guided session.

Referring next to FIG. 8, the technique 800 for respiratory biofeedback-based content selection and playback for a device adjustment is shown. At 802, a RDS is produced using data obtained from one or more sensors of a mobile device running a software application. The RDS represents a stream of respiratory information of the user of the software application while the mobile device rests on the user. The RDS may, for example, include a respiratory curve, a respiratory stability, and a respiratory rate of the user. In some implementations, producing the RDS may include denoising the data obtained using the one or more sensors using a motion noise baseline determined for an environment in which the user is located.

At 804, the RDS is processed to determine a user state of the user of the software application. Processing the respiratory data stream to determine the user state of the user may include classifying one or more of the respiratory curve, the respiratory stability, or the respiratory rate according to user state models to infer the user state, which user state may be an emotional state, a mental state, a physiological state, or a combination thereof. In some implementations, the user state may be determined using the RDS and using biometric user data received from a secondary device in communication with the mobile device.

At 806, functionality of a connected device in communication with the mobile device of a network is triggered based on the user state. In some implementations, rather than the functionality of the connected device being triggered based on the user state, the functionality of the connected device may instead be triggered based on values of the respiratory data stream. The functionality which is triggered in the connected device may generally be functionality modeled to assist with a kind of session being performed or otherwise participated in by the user of the software application. For example, where the user is participating in a meditation session, the functionality may be or refer to the dimming of a brightness level of a smart light, such as a smart lightbulb or a smart light switch. In another example, where the user is participating in a relaxation session, the functionality may be or refer to a change in haptic feedback in a haptic mat or table. In some implementations, the functionality of the connected device may be defined based on a guided session performed or otherwise participated in by the user of the software application.

In some implementations, the connected device may be located in an environment separate from an environment at which the user is located. In such an implementation, triggering the functionality of the connected device, or the technique 800 otherwise, may include signaling an indication of the user state or other aspects of the respiratory information of the user to a person located at that separate environment, such as by the triggering of functionality of a connected device located at that separate environment.

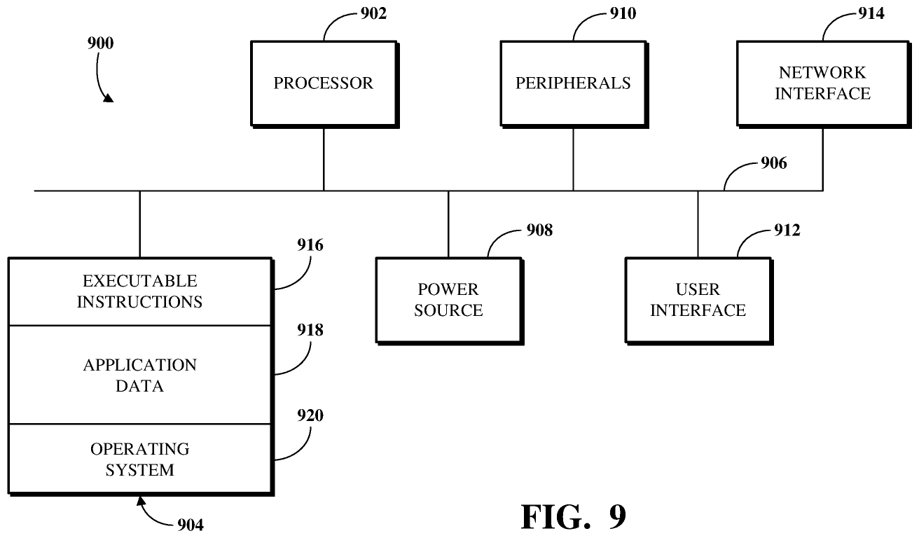

FIG. 9 shows a block diagram of an example internal structure of a computing device 900 which may be used for respiratory biofeedback-based content selection and playback. The computing device 900 may be used to implement a device, for example, the device 100 shown in FIG. 1. Alternatively, the computing device 900 may be used to implement a server on which a software application is run, a client that accesses the software application, and/or another device according to the implementations disclosed herein. The computing device 900 includes components or units, such as a processor 902, a memory 904, a bus 906, a power source 908, peripherals 910, a user interface 912, and a network interface 914. One of more of the memory 904, the power source 908, the peripherals 910, the user interface 912, or the network interface 914 can communicate with the processor 902 via the bus 906.

The processor 902 is a central processing unit, such as a microprocessor, and can include single or multiple processors having single or multiple processing cores. Alternatively, the processor 902 can include another type of device, or multiple devices, now existing or hereafter developed, configured for manipulating or processing information. For example, the processor 902 can include multiple processors interconnected in any manner, including hardwired or networked, including wirelessly networked. For example, the operations of the processor 902 can be distributed across multiple devices or units that can be coupled directly or across a local area or other suitable type of network. The processor 902 can include a cache, or cache memory, for local storage of operating data and/or instructions.

The memory 904 includes one or more memory components, which may each be volatile memory or non-volatile memory. For example, the volatile memory of the memory 904 can be random access memory (RAM) (e.g., a DRAM module, such as DDR SDRAM) or another form of volatile memory. In another example, the non-volatile memory of the memory 904 can be a disk drive, a solid state drive, flash memory, phase-change memory, or another form of non-volatile memory configured for persistent electronic information storage. The memory 904 may also include other types of devices, now existing or hereafter developed, configured for storing data or instructions for processing by the processor 902.

The memory 904 can include data for immediate access by the processor 902. For example, the memory 904 can include executable instructions 816, application data 818, and an operating system 820. The executable instructions 816 can include one or more application programs, which can be loaded or copied, in whole or in part, from non-volatile memory to volatile memory to be executed by the processor 902. For example, the executable instructions 816 can include instructions for performing some or all of the techniques of this disclosure. The application data 818 can include user data, database data (e.g., database catalogs or dictionaries), or the like. The operating system 820 can be, for example, Microsoft Windows®, Mac OS X®, or Linux®; an operating system for a small device, such as a smartphone, tablet device, or wearable device (e.g., a smart watch); or an operating system for a large device, such as a mainframe computer.

In some implementations, the memory 904 can be distributed across multiple devices. For example, the memory 904 can include network-based memory or memory in multiple clients or servers performing the operations of those multiple devices. In some implementations, the application data 818 can include functional programs, such as a web browser, a web server, a database server, another program, or a combination thereof.

The power source 908 includes a source for providing power to the computing device 900. For example, the power source 908 can be an interface to an external power distribution system. In another example, the power source 908 can be a battery, such as where the computing device 900 is a mobile device or is otherwise configured to operate independently of an external power distribution system.

The peripherals 910 includes one or more sensors, detectors, or other devices configured for monitoring the computing device 900 or the environment around the computing device 900. For example, the peripherals 910 can include a geolocation component, such as a global positioning system location unit. In another example, the peripherals can include a temperature sensor for measuring temperatures of components of the computing device 900, such as the processor 902. In some implementations, the computing device 900 can omit the peripherals 910.

The user interface 912 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, or other suitable display.

The network interface 914 provides a connection or link to a network. The network interface 914 can be a wired network interface or a wireless network interface. The computing device 900 can communicate with other devices via the network interface 914 using one or more network protocols, such as using Ethernet, TCP, IP, power line communication, Wi-Fi, Bluetooth, infrared, GPRS, GSM, CDMA, Z-Wave, ZigBee, another protocol, or a combination thereof.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, Swift, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “module” and “component” are used broadly and are not limited to mechanical or physical implementations, but can include software routines in conjunction with processors, etc.

Likewise, the terms “system” or “module” as used herein and in the figures, but in any event based on their context, may be understood as corresponding to a functional unit implemented using software, hardware (e.g., an integrated circuit, such as an ASIC), or a combination of software and hardware. In certain contexts, such systems or modules may be understood to be a processor-implemented software system or processor-implemented software module that is part of or callable by an executable program, which may itself be wholly or partly composed of such linked systems or modules.

Implementations or portions of implementations of the above disclosure can take the form of a computer program product accessible from, for example, a computer-usable or computer-readable medium. A computer-usable or computer-readable medium can be any device that can, for example, tangibly contain, store, communicate, or transport a program or data structure for use by or in connection with any processor. The medium can be, for example, an electronic, magnetic, optical, electromagnetic, or semiconductor device.

Other suitable mediums are also available. Such computer-usable or computer-readable media can be referred to as non-transitory memory or media, and can include volatile memory or non-volatile memory that can change over time. A memory of an apparatus described herein, unless otherwise specified, does not have to be physically contained by the apparatus, but is one that can be accessed remotely by the apparatus, and does not have to be contiguous with other memory that might be physically contained by the apparatus.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for respiratory biofeedback-based content selection and playback, the method comprising:
initiating a guided session at a software application running on a mobile device;
producing, using data obtained using one or more sensors of the mobile device, a respiratory data stream representing a stream of respiratory information of a user of the software application while the mobile device rests on the user;
processing the respiratory data stream to determine a user state of the user;
selecting content for output to the user based on the user state and based on a defined respiratory objective of the guided session;
adjusting initial content previously output to the user during the guided session by outputting the selected content to the user; and
determining a progress of the user toward achieving the defined respiratory objective of the guided session based on a change in the user state resulting from the outputting of the selected content.

2. The method of claim 1, wherein selecting the content for output to the user based on the user state and based on the defined objective of the guided session comprises:
determining, based on at least one of the user state or the defined objective of the guided session, to adjust one or more parameters associated with initial content.

3. The method of claim 2, wherein the initial content includes music output to a speaker, wherein the one or more parameters associated with the initial content correspond to one or both of a volume or tempo of the music.

4. The method of claim 1, further comprising:
determining, based on the respiratory information of the respiratory data stream or the change in the user state, that a transition point of the guided session has been reached by the user; and
adjusting an aspect of the guided session as a result of the user reaching the transition point of the guided session.

5. The method of claim 1, further comprising:
transmitting, to a connected device over a network, a command configured to trigger functionality of the connected device based on the user reaching the transition point of the guided session.

6. The method of claim 5, wherein the connected device is located at a second environment different from an environment in which the user is located during performance of the guided session.

7. The method of claim 1, wherein the respiratory information of the respiratory data stream includes a respiratory curve, a respiratory stability, and a respiratory rate of the user, wherein processing the respiratory data stream to determine the user state of the user comprises:
classifying one or more of the respiratory curve, the respiratory stability, or the respiratory rate according to user state models to infer the user state.

8. The method of claim 1, wherein producing the respiratory data stream comprises:
denoising the data using a motion noise baseline determined for an environment in which the user is located during performance of the guided session.

9. The method of claim 1, further comprising:
responsive to a completion of the guided session, determining a score for the user based on progress by the user toward achieving the defined respiratory objective of the guided session.

10. The method of claim 1, wherein biometric user data is received from a secondary device in communication with the mobile device, wherein processing the respiratory data stream to determine the user state of the user comprises:
using the respiratory data stream and the biometric user data to determine the user state.

11. A method for respiratory biofeedback-based content selection and playback, the method comprising:
processing, during a guided session at a software application running on a mobile device, a respiratory data stream of a user of the software application to determine one or more of a respiratory curve, a respiratory stability, or a respiratory rate of the user;
selecting, based on the one or more of the respiratory curve, the respiratory stability, or the respiratory rate of the user, content to use to adjust initial content previously output to the user during the guided session; and
adjusting the initial content according to the selected content by outputting the selected content during the guided session.

12. The method of claim 11, wherein a biofeedback loop including processing respiratory data streams of the user, selecting new content for output to the user based on the respiratory data streams, and outputting the new content to the user is repeated until the guided session is completed.

13. The method of claim 11, wherein adjusting the initial content according to the selected content comprises:
replacing the initial content with the selected content.

14. The method of claim 11, the selected content is selected from a set of available content items associated with the guided session, wherein ones of the available content items are differently weighted according to a relative value for the guided session.

15. The method of claim 11, wherein the user participates in the guided session while the guided session is lead live by a leader using a leader device.

16. A method for respiratory biofeedback-based content selection and playback, the method comprising:
producing, at a first time and using first data obtained using one or more sensors of a mobile device, a first respiratory data stream of a user of a software application running on the mobile device while the mobile device rests on the user and while the user participates in a guided session of the software application;
processing the first respiratory data stream to select first content to output to the user based on the guided session;
outputting the first content for presentation to the user, wherein the first content is configured to change a user state of the user to a first state;
producing, at a second time after the first time and using second data obtained using the one or more sensors of the mobile device, a second respiratory data stream of the user while the mobile device rests on the user and while the user participates in the guided session;
processing the second respiratory data stream to select second content to output to the user based on the guided session; and
outputting the second content for presentation to the user, wherein the second content is configured to change the user state of the user from the first state to a second state.

17. The method of claim 16, wherein the guided session is a sleep induction guided session or a meditation guided session, wherein the first content is selected based on a first consciousness level associated with an initial state of the user and the second content is selected based on a second consciousness level associated with the first state of the user.

18. The method of claim 17, further comprising:
transmitting, based on the first consciousness level of the user of the software application, a first command to a smart light to cause the smart light to decrease a brightness setting of the smart light to a first level; and
transmitting, based on the second consciousness level of the user of the software application, a second command to the smart light to cause the smart light to decrease the brightness setting of the smart light to a second level.

19. The method of claim 17, wherein further respiratory data streams of the user of the software application are produced and used to output content for presentation to the user until the software application determines the user has fallen asleep or achieved a deep state of consciousness.

20. The method of claim 16, wherein the guided session is a stress or relaxation management guided session, wherein the first content is a cue instructing the user to take deeper breaths, where the second respiratory data stream is measurable to determine a change in breathing by the user after the first content is output for presentation to the user.
